(19) United States
(12) Patent Application Publication
(10) Pub. No.: US 2026/0031245 A1
(43) Pub. Date: Jan. 29, 2026

(54) Methods and Systems to Provide Artificial Intelligence Enhanced Communications Between Healthcare Workers for Patient Care and Documentation

(71) Applicant: T-Mobile Innovations LLC, Overland Park, KS (US)

(72) Inventors: Christina CLAURE, Menlo Park, CA (US); Emma DU, Overland Park, KS (US); Reseda Maria Benitez FLORENTIN, Houston, TX (US); Christine GALL, Waterford, WI (US); Dominic Christopher KLEIN, Lawrence, KS (US); Hai Vy NGO, Kansas City, MO (US); Jay PATEL, Lawrence, KS (US); Isaac Jesse PHILLIPS, Lawrence, KS (US); Yash PRAJAPATI, Lawrence, KS (US); Joao TEIXEIRA, Overland Park, KS (US); Gregory THOMAS, Lawrence, KS (US)

(21) Appl. No.: 18/788,098

(22) Filed: Jul. 29, 2024

(51) Int. Cl. G16H 80/00; G16H 10/60; G16H 50/70

(52) U.S. Cl. CPC G16H 80/00; G16H 10/60; G16H 50/70

(57) ABSTRACT

A healthcare facility system receives medical data associated with a patient from one or more first devices associated with a first healthcare worker, determines determine, using an artificial intelligence model, one or more medical recommendations based on a common pattern identified in the medical data and historical medical data associated with a plurality of prior patients, transmits the one or more medical recommendations to a second device operated by the second healthcare worker, in which each of the one or more medical recommendations represents a task to be performed by the first healthcare worker with respect to the patient, and transmits confirmed medical recommendations to the one or more first devices associated with the first healthcare worker.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

The recent global pandemic has led to nursing shortages worldwide. The actual cause of the nursing shortage is not necessarily the lack of nurses available to work, but instead the senior nurses are increasingly hesitant to work in the conditions during and following the pandemic. Meanwhile, the junior nurses are available and ready to work, but cannot properly be trained due to the shortage of senior nurses. Junior nurses may also sometimes lack sufficient knowledge to manage patients and operate medical equipment. Therefore, the lack of senior nurses with sufficient experience is causing various problems in the healthcare industry.

### SUMMARY

In an embodiment, a method implemented in a communication network including to provide artificial intelligence enhanced communications between healthcare workers for patient care and documentation is disclosed. The method comprises transmitting, by one or more first devices in the communication network, medical data associated with a patient to a healthcare facility system in the communication network, in which the medical data comprises at least one of biometric data of the patient or current symptoms experienced by the patient, and the one or more first devices are operated by a first healthcare worker. The method further comprises identifying, by a routing application of the healthcare facility system, using an artificial intelligence model, a second device operated by a second healthcare worker based on at least one of the medical data associated with the patient, healthcare worker expertise data associated with the second healthcare worker, or healthcare worker capacity data associated with the second healthcare worker, in which the second device is positioned at least a predefined distance away from the one or more first devices. The method further comprises converting, by a record application of the healthcare facility system, the medical data associated with the patient into at least one of text, images, or videos to obtain converted medical data, transmitting, by the record application, the converted medical data to the second device operated by the second healthcare worker, and receiving, by the record application from the second device operated by the second healthcare worker, records comprising data describing at least one of the patient being at least one of admitted into a healthcare facility, tested for one or more conditions, treated for the one or more conditions, medicated with one or more medications, or discharged from the healthcare facility. The method further comprises determining, by a medical application of the healthcare facility system, using the artificial intelligence model, one or more medical recommendations based on a common pattern identified in the medical data and historical medical data associated with a plurality of prior patients, transmitting, by the medical application, the one or more medical recommendations to the second device operated by the second healthcare worker, wherein each of the one or more medical recommendations indicates a task to be performed by the first healthcare worker with regard to the patient, receiving, by the medical application, from the second device operated by the second healthcare worker, a confirmation of at least one of the medical recommendations, and transmitting, by the medical applications, the at least one of the medical recommendations to the one or more first devices operated by the first healthcare worker.

In another embodiment, a method implemented in a communication network including to provide artificial intelligence enhanced communications between healthcare workers for patient care and documentation is disclosed. The method comprises receiving, by a healthcare facility system in the communication network, medical data associated with a patient from one or more first devices associated with a first healthcare worker, in which the medical data comprises at least one of biometric data of the patient or current symptoms experienced by the patient, converting, by a record application of the healthcare facility system, at least a portion of the medical data associated with the patient into text to obtain converted medical data, transmitting, by the record application, the medical data and the converted medical data to a second device operated by a second healthcare worker, and receiving, by the record application from the second device operated by the second healthcare worker, a patient record comprising data describing the patient being at least one of admitted into a healthcare facility, tested for one or more conditions, treated for the one or more conditions, medicated with one or more medications, or discharged from the healthcare facility. The method further comprises determining, by a medical application of the healthcare facility system, using an artificial intelligence model, one or more medical recommendations based on a common pattern identified in the medical data and historical medical data associated with a plurality of prior patients, transmitting, by the medical application to the second device operated by the second healthcare worker, the one or more medical recommendations, in which each of the one or more medical recommendations represents a task to be performed by the first healthcare worker with regard to the patient, receiving, by the medical application, from the second device operated by the second healthcare worker, a confirmation of at least one of the medical recommendations, and transmitting, by the medical application, the at least one of the medical recommendations to the one or more first devices operated by the first healthcare worker.

In yet another embodiment, a healthcare facility system is disclosed. The healthcare facility system comprises a non-transitory memory, a processor coupled to the non-transitory memory, a record application stored at the non-transitory memory, and a medical application stored at the non-transitory memory. The record application, when executed by the processor, causes the processor to be configured to receive medical data associated with a patient from one or more first devices associated with a first healthcare worker, in which the medical data comprises at least one of biometric data of the patient or current symptoms experienced by the patient, transmit the medical data to a second device operated by a second healthcare worker, and receive, from the second device operated by the second healthcare worker, a patient record comprising data describing the patient being at least one of admitted into a healthcare facility, tested for one or more conditions, treated for the one or more conditions, medicated with one or more medications, or discharged from the healthcare facility. The medical application, when executed by the processor, causes the processor to be configured to determine, using an artificial intelligence model, one or more medical recommendations based on a common pattern identified in the medical data and historical medical data associated with a plurality of prior patients, transmit the one or more medical recommendations to the second device operated by the second healthcare worker, in which each of the one or more medical recommendations represents a task to be performed by the first healthcare worker with respect to the patient, and automatically perform at least one of the one or more medical recommendations in response to a confirmation received from the second device to perform the at least one of the one or more medical recommendations.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a block diagram of a communication network according to various embodiments of the disclosure.

FIGS. 2A, 2B, and 2C are a block diagrams illustrating embodiments for providing artificial intelligence (AI) enhanced communications between healthcare workers and patients in the communication network of FIG. 1 according to various embodiments of the disclosure.

FIGS. 3 and 4 are message sequence diagrams illustrating methods for providing AI enhanced communications between healthcare workers and patients in the communication network of FIG. 1 according to various embodiments of the disclosure.

FIG. 5 is a flowchart of a first method for providing AI enhanced communications between healthcare workers and patients according to various embodiments of the disclosure.

FIG. 6 is a flowchart of a second method for providing AI enhanced communications between healthcare workers and patients according to various embodiments of the disclosure.

FIG. 7 is a block diagram of a computer system implemented within the communication system of FIG. 1 according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The less-experienced, junior healthcare workers (e.g., the junior nurses mentioned above) may currently be at a significant disadvantage, due not only to the post-pandemic conditions at healthcare facilities, but also because a large percentage of the more senior healthcare workers (e.g., the senior nurses) have left the medical field due to exhaustion. Said another way, there are significantly fewer senior healthcare workers still working in healthcare facilities after the pandemic, and the ones that are still working in healthcare facilities may no longer be willing to work directly with patients under the current healthcare conditions. Meanwhile, there may be a higher number of junior healthcare workers that are available and willing to work directly with patients under the current healthcare conditions, but these junior healthcare workers may sometimes lack the training that would have otherwise been provided by the senior healthcare workers during the job.

The senior healthcare workers that are available and working may help/train the junior healthcare workers without having direct patient contact by, for example, communicating remotely with one or more junior healthcare workers as the junior healthcare workers work directly with the patient. In this way, the junior healthcare workers are responsible for the direct patient care, while the senior healthcare workers provide guidance to the junior healthcare workers for patient care related tasks. However, healthcare facilities may not be set up to provide efficient and secure network connections between senior healthcare workers and junior healthcare workers to enable the senior healthcare workers to simultaneously work remotely with junior healthcare workers. Moreover, intelligent and secure communication channels that can be used to provide communication channels between not only the healthcare workers but also the medical devices in the patient room have not been developed to enable senior healthcare workers to remotely guide and train junior healthcare workers.

In addition, healthcare workers in general spend more than half their time on documentation tasks as opposed to actual patient care, and this is even more problematic for the emergency department healthcare workers that document all patient records toward the end of their shift (i.e., when it may be difficult for the worker to recall and distinguish between all of the patients of the day). Tools have not been enabled to assist healthcare workers in documentation tasks, medical decisions, training, equipment use, etc., particularly with regard to the communications between remote senior and junior healthcare workers. Therefore, the healthcare industry may experience various technical problems related to the inefficient and ineffective use of computing/radio resources and technologies available in healthcare facilities, which may otherwise be interworked together in an intelligent and resourceful manner to provide more optimal and efficient care to patients.

The present disclosure addresses the foregoing technical problems by providing a technical solution in the technical field of communication systems, and in particular, communication systems used in the healthcare industry. The embodiments disclosed herein are directed to leveraging internal private networks and/or cellular networks with artificial intelligence (AI) models to enable intelligent and secure communications between junior healthcare workers that work directly with the patients and remotely positioned senior healthcare workers. For example, a senior healthcare worker may be positioned at a desk with a computer in a separate room in a hospital, or even external to the hospital, while the junior healthcare worker is actually physically present with the patient. Devices operated/used by the junior health care worker may collect data describing a current condition of a patient, and medical devices may collect biometric data from the patient (e.g., cardiac monitors, sensors, etc.). The devices may send the collected data to a device of a senior healthcare worker over a radio connection (e.g., using a radio transceiver). The senior healthcare worker may use the collected data to perform documentation tasks remotely while the junior healthcare worker focuses on the patient care related tasks. The data may also be provided to an AI model to generate routing, record, and medical recommendations, as further described herein. Therefore, the embodiments disclosed herein resolve the aforementioned technical problems by utilizing the computing/radio resources and technologies available to the healthcare workers within and external to healthcare facilities to provide more optimal and efficient care to patients.

The patient may be located at a medical site (e.g., medical room in a healthcare facility, emergency vehicle, patient home, etc.), and a junior healthcare worker (e.g., nurse) may be with the patient at the medical site. Meanwhile, a senior healthcare worker may be positioned away from the junior healthcare worker and the patient (e.g., at least a predefined distance away from the junior healthcare worker/patient, in another room within the healthcare facility or external to the healthcare facility). The junior healthcare worker may operate one or more first devices (e.g., a portable handheld computing device, a wearable computing device, etc.), while the senior healthcare worker operates at least one second device (e.g., a computer, laptop, tablet, etc.). A communication network may include both the first devices and the second device, and may also include a healthcare facility system, one or more medical devices configured to sense data related to the patient, one or more data stores and an AI model, all interconnected via a network.

In an embodiment, one or more of the first devices operated by the junior healthcare worker may obtain medical data associated with a patient. For example, a first device may be a computing device embodied as a wearable lapel, which may include a microphone and a radio transceiver, which may capture a recording of the conversation between the junior healthcare worker and the patient, and transmit the recording to the healthcare facility system. Another first device may be a computer or tablet, and the junior healthcare worker may manually enter current patient data describing the current symptoms experienced by the patient into the first device via a user interface, and then transmit the current patient data to the healthcare facility system. The first device may also receive medical device data from the different medical devices that may be hooked up to or used on the patient. For example, the medical devices may include a camera, a sensor, a cardiac monitor, a defibrillator, an oxygen delivery system, computed tomography scanner, a suction unit, airway management equipment, a splinting and immobilization device, first aid supplies, intravenous supplies, diagnostic equipment and/or various types of equipment. The medical device data obtained from the different medical devices may include, for example, biometric information and/or vital signs (e.g., heart rate, blood pressure, respiratory rate, temperature). Each of the medical devices may also include a processor and a radio transceiver, to obtain different types of data, images, videos, etc., and transmit the data to a first device or directly to the healthcare facility system. The first device may package the medical data (including the current patient data and the medical device data) together and transmit the medical data to the healthcare facility system periodically or continuously via a radio connection, as medical data is collected at the healthcare facility system.

The healthcare facility system may include a routing application that may use an AI model (e.g., machine learning model, neural networking model, learning action model, etc.) to dynamically route the medical data to a second device operated by an optimal, intelligently selected senior healthcare worker. For example, the AI model may be trained with data describing all the healthcare workers associated with a particular healthcare facility system. Each healthcare facility system may be associated with a healthcare facility and/or a healthcare worker group, and in this way, the AI model may be trained with data describing healthcare workers working in the healthcare facility or employed with the healthcare worker group. The data describing the healthcare workers may include, for example, worker expertise data describing the expertise, specialty, and level of experience of each healthcare worker associated with the healthcare facility system. The data describing the healthcare workers may also include worker capacity data describing whether a healthcare worker has the availability, time, and resource capacity to work with another healthcare worker or a patient. The data describing the healthcare workers may also include worker data defining a patient interaction history with the healthcare workers. The AI model may be continuously updated/trained as the worker expertise data, worker capacity data, and worker data is updated at the healthcare facility system.

Once the AI model has been trained, the routing application may use the AI model to identify a second device operated by a senior healthcare worker based on the medical data received from the first device. The senior healthcare worker may be optimally identified based on the expertise of the senior healthcare worker (as indicated in the worker expertise data), the capacity of the senior healthcare worker (as indicated in the worker capacity data), and whether the patient has a history with the senior healthcare worker (as indicated in the worker data).

The healthcare facility system may also include a record application that may process the medical data received from the first device as needed. For example, the record application may transform the medical data from one format to another format for consistency or normalization purposes. The record application may use other AI tools to convert at least a portion of the medical data (e.g., convert an audio recording of a conversation between the junior healthcare worker and the patient into text using, for example, voice biometrics tools and/or speech-to-text algorithms provided by the AI model). The record application may then transmit the medical data (including the transformed/converted portions of the medical data) to the identified second device operated by an optimal senior healthcare worker.

The second device of the senior healthcare worker may receive the medical data (again including the transformed/converted portions of the medical data) and display the medical data in a human-viewable format (e.g., via an application or a webpage) on a display of the second device (e.g., display text, play records, play videos, present scans/images, etc.). In some cases, the senior healthcare worker may use the displayed medical data to generate all the patient records for a patient being seen by a junior healthcare worker. In this way, the junior healthcare worker may not need to manually create the patient records for the patient at a later time; instead the documentation tasks may be handled by the senior healthcare worker, sometimes in parallel while the junior healthcare worker is seeing the patient, thereby drastically increasing the efficiency of documentation tasks in the healthcare industry. The patient records may include, information describing various aspects of patient care, including, for example, data describing the patient being admitted into the healthcare facility, tests performed on the patient, medicines provided to the patient, treatments provided to the patient, symptoms/side effects/discomfort, etc. experienced by the patient, the patient being discharged from the healthcare facility, etc.

For example, the junior healthcare worker may be listening to the patient describe current symptoms, a wearable device (e.g., first device) of the junior healthcare worker may record this conversation, and the record application may send the recording or a text document transcribing the recording to the identified senior healthcare worker in real-time. The senior healthcare worker may manually generate patient notes describing the symptoms experienced by the patient as the patient is describing the symptoms to the junior healthcare worker, such that the junior healthcare worker need not separately document any information gleaned during the conversation with the patient (thereby allowing the junior healthcare worker to spend more time and focus on actual patient care instead of documentation tasks).

In another case, the record application may use the AI model to make record recommendations suggesting information that might be relevant to a patient record (e.g., suggested admission summaries or discharge summaries, suggested human-readable text versions of the medical device data directly, etc.). The record application may transmit the record recommendations to the second device of the senior healthcare worker. The second device may display the record recommendations, and the senior healthcare worker may view the information in the record recommendations and then confirm whether to add the record recommendations into a patient record or not. When confirmed, the second device may use the AI model to automatically insert the information in the record recommendation into a patient record, and/or store the patient record in a data store of the healthcare facility system. The confirmations/rejections of the record recommendations may be used to further train the AI model to generate more accurate recommendations over time.

The healthcare facility system may also include a medical application that may use the AI model to generate medical recommendations or perform various tasks/actions in response to receiving a confirmation/command from the senior healthcare worker. For example, the AI model may be trained with historical medical data for multiple prior patients that have been cared for by one or more healthcare facilities associated with a healthcare facility system. The historical medical data may include, for example, symptoms data describing symptoms experienced by the prior patients, diagnosis data describing tests conducted on the prior patients and the actual medical diagnosis of the prior patients, treatment data describing medications provided to the prior patients or treatments provided to the prior patients that may or may not have successfully provided relief to the prior patients. The AI model may also be trained with general medical education data that may supplement the historical medical data specifically pertaining to the prior patients. The AI model may use the medical education data and the historical medical education data to identify certain patterns or trends, which may be used to make predictions/recommendations for medical strategies for a current patient.

In an embodiment, the medical application may obtain the current patient data (describing a current state and conditions of the patient) and the medical device data (from the medical data received by the healthcare facility system), and input this data into the AI model to generate one or more medical recommendations (e.g., based on the current patient data, the medical device data, and the patterns/trends identified by AI model). For example, the medical recommendations may include a recommendation to prescribe/administer a particular medicine at a predefined dose to a patient, a recommendation to prescribe/administer a newer more effective version of a previously prescribed medicine to a patient, a recommendation to turn the patient to one side for pain relief, a recommendation on an easier/safer method to perform a procedure or task with respect to a patient, a recommendation to consult a specialist for a medical condition, and/or any other type of recommendation that may be relevant to the care and maintenance of the patient.

The medical application may transmit, via a radio connection, the medical recommendations for display at the second device of the senior healthcare worker. The senior healthcare worker may view the medical recommendations and then confirm whether to accept or reject the medical recommendations. For example, the senior healthcare worker may review a medical recommendation to turn the patient on to his/her left side for pain relief, and confirm the medical recommendation by selecting a user interface button or icon presented on the display of the second device. The medical application may then send the confirmed medical recommendation and/or instructions pertaining to the confirmed medical recommendation to one of the first device operated by the junior healthcare worker, in which the medical recommendation/instructions may be presented for display at the first device. In this example, text suggesting that the junior healthcare worker turn the patient over on to his/her left side may be presented on a display of the first device. The first device may also display one or more user interface buttons, which may be selected by the junior healthcare worker when the healthcare worker completes the task indicated in the instructions.

In an embodiment, the AI model may be trained to output various types of recommendations, which may be confirmed/rejected by the senior healthcare worker, and actually performed by the junior healthcare worker when confirmed. For example, the AI model may be trained with equipment data, which may include instruction manuals or instructions provided by other healthcare workers for operating various types of equipment (including the aforementioned medical devices) that may be used in association with the patient. For example, the equipment data may include IV line instruction, central line instructions, CT scanner instructions, cardiac monitor instructions, etc. In some cases, the medical recommendations may include the use of one or more of these types of equipment, and the junior healthcare worker may not have experience operating the equipment indicated in the medical recommendation. In this case, the medical application may input the selected/confirmed medical recommendations into the AI model to generate (patient-specific) step-by-step instructions for operating the equipment to perform the task indicated in the medical recommendation. The medical application may then the send step-by-step instructions to one of the first devices operated by the junior healthcare worker, in which the step-by-step instructions may be presented for display at the first device. In this example, the step-by-step instructions may be in the form of text, images, and/or videos. The first device may display the step-by-step instructions and one or more user interface buttons, which may be selected by the junior healthcare worker when the healthcare worker completes the task indicated in the medical recommendation.

In some cases, the AI model may also be trained with general medical education data (i.e., not specific to a particular patient). The medical application may input the selected/confirmed medical recommendations into the AI model to retrieve relevant, patient-specific medical education data that might be informative to the junior healthcare worker while performing the task indicated in the medical recommendation. The medical application may then send the relevant medical education data to one of the first devices operated by the junior healthcare worker, in which the relevant medical education may be presented for display at the first device. In this example, the relevant medical education may also be in the form of text, images, and/or videos.

The connection between the senior and junior healthcare workers may also be used for general two-way communications, either by voice, text, messaging, file exchanges, or any other form of communications. For example, the first device may receive alarms based on the medical device data (e.g., vital signs/biometrics) that is received in association with the patient. The alarms may be triggered when the medical device data meets a condition or crosses a threshold related to a medical emergency associated with the patient (e.g., indicating a cardiac emergency or other type of medical emergency), which may require immediate attention by the junior healthcare worker. The alarms may also be forwarded to the second device of the senior healthcare worker. The senior and junior healthcare workers may communicate using the established channels based on the medical device data and alarms to provide immediate and accurate patient care.

In this way, the embodiments disclosed herein serve to create intelligent and secure radio communication connections between a senior, remote healthcare worker, one or more junior healthcare workers with direct access to a patient, medical devices, and a healthcare facility system. The disclosed multi-channel connections between a senior healthcare worker and one or more junior healthcare workers enable patient records and patient/equipment-related data to be generated and stored in a far more effective and resource efficient manner. The embodiments disclosed herein also enable data received from medical devices, historical patient data, medical education data, equipment instruction manuals, and other types of data stored across various remote locations to be used together via the AI model to provide optimal medical care to a patient, while decreasing data redundancies and increasing resource efficiencies. Therefore, in general, the embodiments disclosed herein also serve to increase healthcare system capacity by decreasing medical errors and increasing medical equipment use efficiency, while providing a work environment for senior healthcare workers to train junior healthcare workers and provide patient care, without actually working directly with the patients.

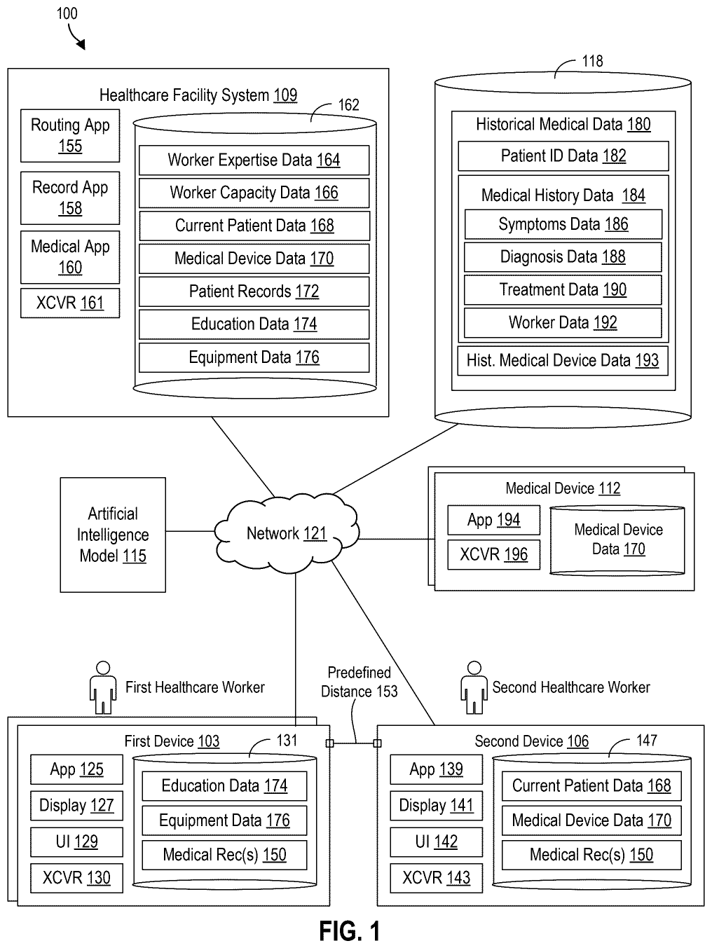

Turning now to FIG. 1, a communication network 100 is described. The communication network 100 includes one or more first devices 103, one or more second devices 106, a healthcare facility system 109, one or more medical devices 112, an AI model 115, a data store 118, and a network 121. Network 121 may be one or more private networks, one or more public networks, or a combination thereof, interconnecting the devices 103, 106, healthcare facility system 109, medical devices 112, AI model 115, and data store 118. While FIG. 1 illustrates the healthcare facility system 109, AI model 115, and data store 118 as being separate from the network 121, it should be appreciated that in some embodiments, the healthcare facility system 109, AI model 115, and data store 118 may be part of the network 121. While FIG. 1 illustrates the healthcare facility system 109 as being separate from the data store 118, it should be appreciated that in some embodiments, the healthcare facility system 109 may include the data store 118. While FIG. 1 illustrates the AI model 115 as being separate from the healthcare facility system 109, it should be appreciated that in some embodiments, the AI model 115 may be included as part of the healthcare facility system 109.

The first device 103 may be operated by a junior healthcare worker (also referred to herein as a “first healthcare worker”). As mentioned above, the junior healthcare worker may have less experience than senior healthcare workers, and may work directly with the patient to provide patient care based on instructions/recommendations provided by the senior healthcare workers. The first device 103 may be, for example, a mobile phone, tablet, personal computer, wearable device, or any other device that includes one or more components such as a display 127, a user interface 129, a radio transceiver 130 (shown in FIG. 1 as “XCVR 130”), a microphone, a speaker, a camera, a processor, a memory, etc. The radio transceiver 130 may be a cellular transceiver configured to establish a wireless communication link with a cell site in the communication network 100 according to a 5G, a long-term evolution (LTE), a code division multiple access (CDMA), or a global system for mobile communication (GSM) telecommunication protocol. The radio transceiver 130 may also support relatively short-range radio communication, and for example, may be embodied as a WiFi radio transceiver, a Bluetooth radio transceiver, or another short-range radio transceiver. The junior healthcare worker may use multiple different first devices 103 simultaneously. For example, the junior healthcare worker may be wearing a wearable first device 103 (e.g., watch or lapel), which may include a camera and a microphone, and may operate a computer (e.g., another first device 103) positioned within a patient room at the same time.

For example, the first device 103 may be implemented as a computer system 700. The first device 103 may include an application 125 for receiving various types of data directly from the junior healthcare worker (e.g., via the user interface 129), from the medical devices 112, or even from other external data stores, and sending this data to the appropriate entity. The different types of data received by the first device 103 may include (patient-specific) education data 174, (patient-specific) equipment data 176, and the medical recommendations 150, each of which may be stored in a data store 131 (e.g., one or more memories) of the first device 103. The education data 174 may refer to patient-specific medical education data 174, which may be received from an external education-related data store or the AI model 115, and may in some cases be presented to the junior healthcare worker to assist in patient care. The (patient-specific) equipment data 176 may refer to instructions, settings, or configurations that may be presented to the junior healthcare worker to assist in using various types of medical equipment with respect to a patient. The medical recommendations 150 are the medical-related recommendations generated by the healthcare facility system 109 using the AI model 115, as further described herein.

The second device 106 may be operated by a senior healthcare worker (also referred to herein as a “second healthcare worker”) who is positioned remotely from the junior healthcare worker and the patient (e.g., at least a predefined distance 153 from the junior healthcare worker). For example, the senior healthcare worker may be positioned in a different room or office within a healthcare facility or external to the healthcare facility (e.g., at home). The second device 106 may be, for example, a mobile phone, tablet, personal computer, or any other device that includes one or more components such as a display 141, a user interface 142, a radio transceiver 143 (shown in FIG. 1 as “XCVR 143”), a microphone, a speaker, a camera, a processor, a memory, etc. The radio transceiver 143 may be a cellular transceiver configured to establish a wireless communication link with a cell site in the communication network 100 according to a 5G, a LTE, a CDMA, or a GSM telecommunication protocol. The radio transceiver 143 may also support relatively short-range radio communication, and for example, may be embodied as a WiFi radio transceiver, a Bluetooth radio transceiver, or another short-range radio transceiver.

For example, the second device 106 may be implemented as a computer system 700. The second device 106 may include an application 139 for receiving various types of data from the healthcare facility system 109 and other sources in the communication network 100. The different types of data received by the second device 106 includes the current patient data 168, medical device data 170, and the medical recommendations 150, each of which may be stored in a data store 147 (e.g., one or more memories) of the second device 106 or communicatively coupled to the second device 106. The current patient data 168 may include data describing a current state or condition of the patient as recorded by the junior healthcare worker (e.g., symptoms currently experienced by the patient, patient identification information, etc.) For example, the current patient data 168 may include data (e.g., recording or text) of a conversation between the junior healthcare worker and the patient. The medical device data 170 may include data received from the medical devices 112 positioned with respect to the patient, in some cases, hooked up to the patient or performing a diagnostic procedure on the patient. For example, the medical device 170 may include biometric information and/or vital signs (e.g., heart rate, blood pressure, respiration rate, temperature).

The healthcare facility system 109 may be a computer system, server software/hardware, or a collection of processors, memories, and/or networking resources, used to manage, receive, and transmit different types of data as described herein. For example, each healthcare facility system 109 may be embodied as a cloud-based system, which may include one or more data stores and memories located together or separately across geographically disparate locations, separate from the respective healthcare facility or group of healthcare workers. Each healthcare facility system 109 may also be embodied as a local set of data stores and memories positioned within or proximate to a respective healthcare facility. A healthcare facility may be, for example, a hospital emergency department, trauma center, cardiac center, stroke center, maternity hospital, psychiatric hospital, rehabilitation center, specialty hospital, urgent care center, long-term care facility, etc. A single healthcare facility may employ multiple different groups of healthcare workers, each contracted with a separate organization. Nevertheless, the healthcare facility system 109 may maintain data related to multiple different groups of healthcare workers.

The healthcare facility system 109 may include a routing application 155, a record application 158, a medical application 160, a radio transceiver 161, and a data store 162. The radio transceiver 161 may be a cellular transceiver configured to establish a wireless communication link with a cell site in the communication network 100 according to a 5G, a LTE, a CDMA, or a GSM telecommunication protocol. The routing application 155, record application 158, and medical application 160 may each be instructions stored across one or more memories, which may be executed by a processor of the healthcare facility system 109 to perform the steps described herein. The routing application 155 may dynamically connect one or more junior healthcare workers (i.e., first devices 103) to an optimal senior healthcare worker (e.g., second device 106), as further described herein. The record application 158 may facilitate use of the data received by the first device 103 and from the medical devices 112 to generate and store patient records 172, as further described herein. The medical application 160 may generate medical recommendations 150 using the AI model 115 based on various types of data, as further described herein.

The data store 162 may be a collection of one or more memories (distributed or co-located) for storing various types of data. While the data store 162 is shown in FIG. 1 as being part of the healthcare facility system 109, it should be appreciated that the data store 162 may be external to the healthcare facility system 109. The data store 162 may store data related to the healthcare workers associated with the healthcare facility system 109 (e.g., employed by, contracted with, or using the resources of the healthcare facility system 109 or an associated healthcare facility). For example, the data store 162 may store worker expertise data 164 and worker capacity data 166. The worker expertise data 164 may include data describing the expertise, specialty, and level of experience of each healthcare worker associated with the healthcare facility system 109. The worker capacity data 166 may include data describing whether a particular healthcare worker has the availability, time, and resource capacity to work with another healthcare worker or a patient. For example, a record may be stored at the data store 162 for each healthcare worker based on an identification of each healthcare worker, and each record may include the corresponding worker expertise data 164 and worker capacity data 166 specific to the respective healthcare worker.

The data store 162 may also store data collected from the first devices 103 and the medical devices 112. For example, the data store 162 may store the current patient data 168, which as described above includes data describing a current state or condition of a patient. The data store 162 may also store medical device data 170 received from one or more medical devices 112 via a radio connection.

The data store 162 may also store patient records 172, which may be received from the second device 106. The patient records 172 may include various types of documented data associated with a patient. For example, a patient record 172 for a patient may include comprehensive patient demographics, medical history, clinical assessments, diagnosis and treatment plans, progress notes detailing the patient's condition and response to treatment, nursing care plans outlining interventions and monitoring, medication administration records, consents/legal documents, discharge planning details, and communication logs among healthcare providers.

The data store 162 may also store general medical education data 174, which may include information used for training purposes, including curriculum content and teaching methodologies. The data store 162 may also include equipment data 176, which may include data from instruction manuals of different types of medical equipment and/or instructions provided by other healthcare workers for operating various types of equipment. While FIG. 1 depicts the education data 174 and the equipment data 176 as being stored in the data store 162 of the healthcare facility system 109, it should be appreciated that the education data 174 and the equipment data 176 may be stored in a data store external to the healthcare facility system 109.

The medical devices 112 refer to medical equipment, tools, or devices, which may be used to collect biometric data of a patient, vital signs of a patient, and/or data describing a current state or condition of the patient. For example, the medical devices 112 may include a camera, a sensor, a cardiac monitor, a defibrillator, an oxygen delivery system, computed tomography scanner, a suction unit, airway management equipment, a splinting and immobilization device, first aid supplies, intravenous supplies, diagnostic equipment and/or various types of equipment. The medical devices 112 may each include an application 194 for collecting/processing medical device data 170 and a radio transceiver 196 for transmitting the medical device data 170 to the healthcare facility system 109/second device 106. The radio transceiver 196 may be a cellular transceiver configured to establish a wireless communication link with a cell site in the communication network 100 according to a 5G, a LTE, a CDMA, or a GSM telecommunication protocol. The radio transceiver 196 may also support relatively short-range radio communication, and for example, may be embodied as a WiFi radio transceiver, a Bluetooth radio transceiver, or another short-range radio transceiver. The medical device data 170 may include, for example, biometrics, vital signs, scanned images, X-ray images, blood test results, cardiac readings, etc., each indicative of a current medical condition of the patient.

The data store 118 may be a collection of one or more memories (distributed or co-located) for storing historical data associated with prior patients treated using the resources of the healthcare facility system 109 and healthcare workers associated with the healthcare facility system 109. The data store 118 may include historical medical data 180 describing multiple prior patients and a medical history of each of the prior patients. The historical medical data 180 for a prior patient may include patient identification data 182 (e.g., name, address, birth date, etc. of a patient), medical history data 184, and historical medical device data 193 of the patient. The medical history data 184 of the patient may include symptoms data 186, diagnosis data 188, treatment data 190, and worker data 192. The symptoms data 186 may include data describing symptoms experienced by the patients, the diagnosis data 188 may include data describing tests conducted on the patients and the actual medical diagnosis of the patients, and the treatment data 190 may include data describing medications provided to the patients or treatments provided to the patients that may or may not have successfully provided relief to the patients. The worker data 192 may include data describing or identifying the healthcare workers that have previously met or have a history of working with particular patients. The historical medical device data 193 may include medical device data 170 previously received for each of the prior patients.

The AI model 115 may be a computer system (e.g., including both software and hardware components) designed to make predictions or forecasts (e.g., the medical recommendations 150 and/or record recommendations) based on patterns or trends learned from historical data (e.g., historical medical data 180 and historical medical device data 193). The AI model 115 may be implemented using software (e.g., algorithms, logic, and code) stored across memories. The host of the AI model 115 (which may be an external server or the healthcare facility system 109) may provide the computational resources for execution of the AI model 115. AI model 115 may be implemented as one or more different types of models using, for example, linear regression, decision trees, support vector machines, neural networks, or ensemble methods. The AI model 115 may be a machine learning model, deep learning model, neural networking model, natural language processing (NLP) model, learning action model, or any other type of AI model. It should be appreciated that any type of AI/predictive model may be used, and the underlying algorithms, computations, and machine learning libraries used by the AI model 115 should not be limited herein.

The AI model 115 may be trained using the historical medical data 180 and historical medical device data 193 of prior patients, worker expertise data 164, worker capacity data 166, education data 174, equipment data 176, other known data, such that the data points and algorithms in the AI model 115 may be used to identify patterns/trends to predict the medical recommendations 150, record recommendations, or other recommendations. The AI model 115 may also be trained to determine a confidence score for each of the predicted recommendations, such that a recommendation is only sent to the second device 106 when the confidence score of the recommendation exceeds a threshold. In some cases, the AI model 115 may also include speech-to-text algorithms, voice biometrics, and other voice verification algorithms, which may be used to translate speech in a recording (video or audio) to text.

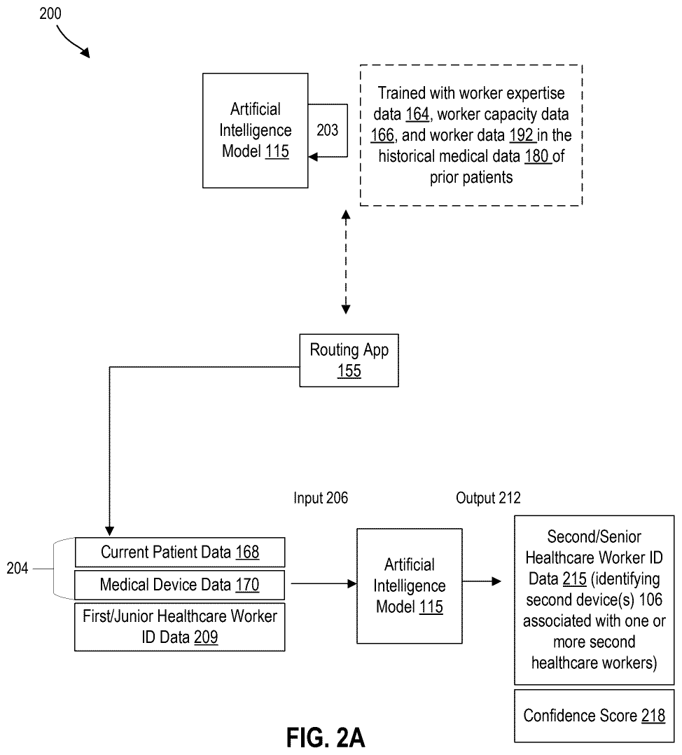

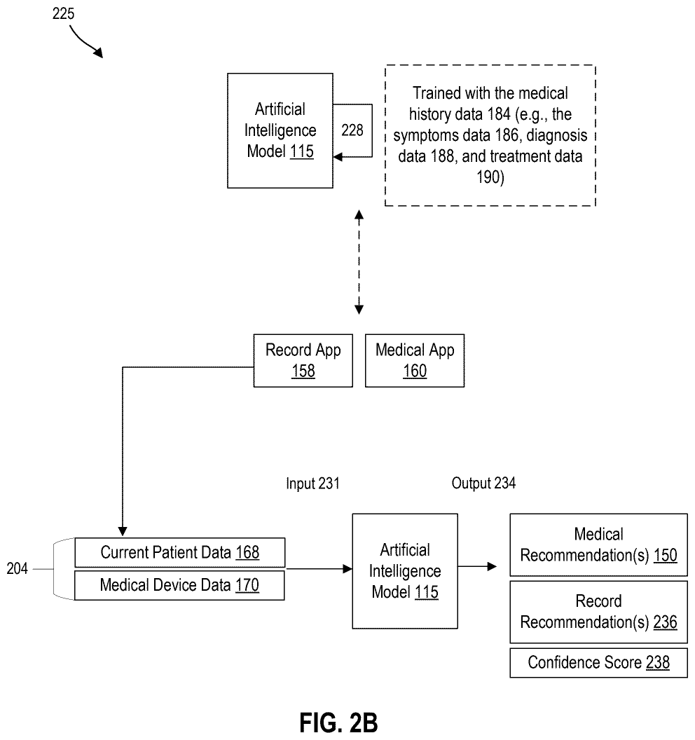

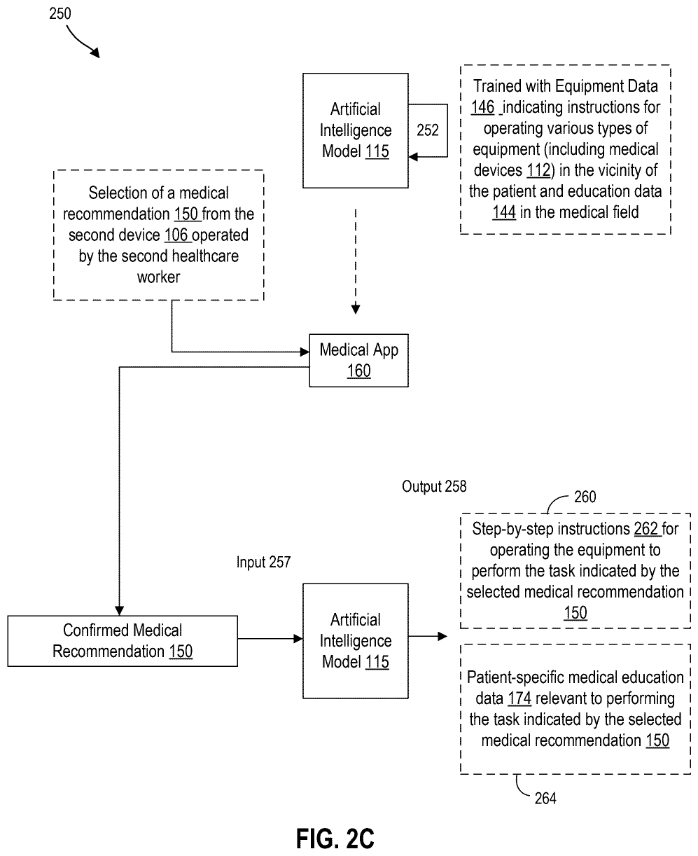

Turning now to FIGS. 2A-2C, shown are block diagrams illustrating the use of the AI model 115 to enhance communications between healthcare workers for patient care and documentation. Specifically, FIG. 2A illustrates the use of the AI model 115 to identify an optimal senior healthcare worker to connect with a junior healthcare worker, FIG. 2B illustrates the use of the AI model 115 to generate recommendations (medical recommendations 150 and record recommendations), and FIG. 2C illustrates the use of the AI model 115 to generate instructions for equipment operations and to provide relevant medical education data to a junior healthcare worker.

Referring specifically now to FIG. 2A, shown is a block diagram illustrating the training of the AI model 115 and the use of the AI model 115 to identify an optimal senior healthcare worker to connect with a junior healthcare worker. FIG. 2A specifically illustrates a method 200 performed by the routing application 155 of the healthcare facility system 109 to identify an optimal senior healthcare worker to connect with a junior healthcare worker.

Method 200 may begin with operation 203 to train the AI model 115 using the worker expertise data 164, worker capacity data 166, and/or worker data 192. For example, the routing application 155 may periodically or continuously transmit updated worker expertise data 164 and worker capacity data 166 to the AI model 115 to continuously update the training, weights, and/or prediction algorithms of the AI model 115. The routing application 155 may also instruct the data store 118 to periodically send updates of the worker data 192 to the AI model 115, such that the AI model 115 may correspondingly update the training, weights, and/or prediction algorithms of the AI model 115. Therefore, the AI model 115 is trained based on the expertise and specialty of all healthcare workers associated with a healthcare facility system 109 (e.g., as indicated in the worker expertise data 164), the availability and capacity of the healthcare workers (e.g., as indicated in the worker capacity data 166), a history between different patients and different healthcare workers (e.g., as indicated in the worker data 192).

After and as the AI model 115 is continuously trained, the routing application 155 may use the AI model 115 to identify a senior healthcare worker to connect with the junior healthcare worker. One or more first devices 103 of the junior healthcare worker may collect medical data 204, which may include the medical device data 170 (received from the medical devices 112 over a radio connection) and the current patient data 168 (received from the junior healthcare worker via the user interface 129). The first devices 103 may transmit this medical data 204 to the routing application 155 at the healthcare facility system 109. The junior healthcare worker may also provide a junior healthcare worker identification data 209 (shown in FIG. 2A as “first/junior healthcare worker identification data”) to the first device 103 via the user interface 129, and the first device 103 may transmit the junior healthcare worker identification data 209 to the routing application 155. The junior healthcare worker identification data 209 may include, for example, a name, employee identification number, date of birth, etc. of the junior healthcare worker.

The routing application 155 may provide the medical data 204 and the junior healthcare worker identification data 209 as input 206 into the AI model 115. The AI model 115 may run various methods/algorithms, developed based on the aforementioned training, to generate senior healthcare worker identification data 215 (shown in FIG. 2A as “second/senior healthcare worker identification data”) as output 212. The senior healthcare worker identification data 215 may include an identifier (e.g., name, employee identification number, date of birth, etc.) of the identified senior healthcare worker. The senior healthcare worker may be identified as the optimal senior healthcare worker to connect with the junior healthcare worker based on, for example, an identified match between the medical data 204 (current patient data 168 and medical device data 170) of the patient and the worker expertise data 164 of the senior healthcare worker. In addition or alternatively, the senior healthcare worker may be identified as the optimal senior healthcare worker to connect with the junior healthcare worker based on, for example, the senior healthcare worker having previously cared for the patient (e.g., as indicated in the worker data 192). The senior healthcare worker may also be identified based on the availability of the senior healthcare worker to provide immediate or timely assistance to the junior healthcare worker.

In an embodiment, the output 212 of the AI model 115 may also include a confidence score 218 associated with the senior healthcare worker identification data 215. The confidence score 218 may be a value (e.g., between 0 to 1) representing a confidence or likelihood that the predicted senior healthcare worker is a good match to train the junior healthcare worker while providing optimal care for the patient. In some cases, the greater the number of data points indicating a history of the predicted senior healthcare worker being an optimal match for a junior healthcare work and patient, the higher the confidence score 218 of the senior healthcare worker.

In an embodiment, when the confidence score 218 is greater than a predefined threshold, the routing application 155 may initiate a radio connection between the second device 106 of the senior healthcare worker and a first device 103 of the junior healthcare worker using the radio transceiver 143 of the second device 106 and the radio transceiver 130 of the first device 103. For example, the routing application 155 may transmit instructions and the connection information (e.g., identifiers, usernames, phone numbers, etc. of the first device 103) to the second device 106 to initiate a messaging, video, and/or voice call connection with the first device 103. For example, once the connection is initiated, the application 125 running on the first device 103 may display a window on a display 127 of the first device 103, and the application 139 running on the second device 106 may display a window on a display 141 of the second device 106.

The senior healthcare worker may transmit, from the second device 106 using the radio transceiver 143, feedback data to the AI model 115 indicating whether the senior healthcare worker was indeed an optimal match with the junior healthcare worker and/or the patient. For example, the feedback data may indicate a value (e.g., between 0 and 1) to validate the accuracy of the predicted senior healthcare worker or invalidate the accuracy of the predicted senior healthcare worker. The AI model 115 may use the received feedback data to adjust the training, weighting, and/or prediction algorithms to improve the system to generate more accurate predictions.

Referring specifically now to FIG. 2B, shown is a block diagram illustrating the training of the AI model 115 and the use of the AI model 115 to generate recommendations (medical recommendations 150 and record recommendations 236) for transmission to a second device 106 of a senior healthcare worker. FIG. 2B specifically illustrates a method 225 performed by the record application 158 and/or the medical application 160 of the healthcare facility system 109 to generate recommendations (medical recommendations 150 and record recommendations 236) for transmission to a second device 106 of a senior healthcare worker.

Method 225 may begin with operation 228 to train the AI model 115 using the historical medical data 180 (e.g., the medical history data 184) of multiple prior patients. For example, the medical application 160 may periodically or continuously transmit instructions to the data store 118 to forward, to the AI model 115, updates to the historical medical data 180. The AI model 115 may use the updates to continuously update the training, weighting, and/or prediction algorithms of the AI model 115. The historical medical data 180 stored in the data store may include data describing the medical history and medical care of tens, hundreds, thousands, or even millions of prior patients. The more known data included in the historical medical data 180 that is used to train the AI model 115, the more accurate the predictions of the AI model 115. Therefore, the AI model 115 is trained based on the correlations, patterns, and trends between symptoms (e.g., as indicated in the symptoms data 186), diagnoses (e.g., as indicated in the diagnosis data 188), and treatment (e.g., as indicated in the treatment data 190) identified in the medical history data 184 of a vast amount of prior patients.

After and as the AI model 115 is continuously trained, the medical application 160 may use the AI model 115 to generate various types of recommendations to transmit to the second device 106 of the senior healthcare worker for confirmation or rejection. As mentioned above, one or more first devices 103 of the junior healthcare worker may collect medical data 204, which may include the medical device data 170 and the current patient data 168, and the first devices 103 may transmit this medical data 204 to the record application 158 and/or the medical application 160. The record application 158 and/or the medical application 160 may provide the medical data 204 as input 231 into the AI model 115. The AI model 115 may run various methods/algorithms, developed based on the aforementioned training, to generate various types of recommendations, including medical recommendations 150 and record recommendations 236, as output 234. For example, the medical application 160 may use the AI model 115 to obtain (e.g., generate or receive) medical recommendations 150, while the record application 158 may use the AI model 115 to obtain record recommendations 236.

A medical recommendation 150 may include medical diagnosis or treatment recommendations for a patient (e.g., a diagnosis-related, testing-related, treatment-related, drug-related, etc.), generated based on the correlations, patterns, and trends identified in the medical history data 184. For example, the medical recommendation 150 may include recommendation to prescribe/administer a particular medicine at a predefined dose to a patient, a recommendation to prescribe/administer a newer more effective version of a previously prescribed medicine to a patient, a recommendation to turn the patient to one side for pain relief, a recommendation on an easier/safer method to perform a procedure or task with respect to a patient, a recommendation to consult a specialist for a patient, and/or any other type of recommendation that may be relevant to the care and maintenance of the patient.

A record recommendation 236 may include a recommendation to include certain portions of the medical data 204 or deductions inferred from the medical data 204 (e.g., based on the correlations, patterns, and trends identified in the medical history data 184 and medical data 204) in a patient record 172 of a current patient. For example, the record recommendation 236 may include suggested admission summaries or discharge summaries, suggested human-readable text versions of the medical device data directly, etc.

In an embodiment, the output 234 of the AI model 115 may also include a confidence score 238 associated with each generated medical recommendation 150 and record recommendation 236. The confidence score 238 may be a value (e.g., between 0 to 1) representing a confidence or likelihood that the predicted recommendation is accurate. In some cases, the greater the number of data points indicating a history of accurately predicted recommendations 150 and 236 (e.g., based on whether the second healthcare confirmed or rejected the recommendation 150 and/or 236), the higher the confidence score 238 of the predicted recommendations 150 and 236.

In an embodiment, when the confidence score 238 is greater than a predefined threshold, the record application 158 may transmit the record recommendation 236 to the second device 106 of the senior healthcare worker. The application 139 at the second device 106 may present the record recommendation 236 (e.g., in human-readable or viewable form) for display on the display 141. The senior healthcare worker may select a user interface button/icon on the user interface 142 to either confirm the record recommendation 236 (e.g., to indicate that the record recommendation 236 is accurate and is to be included in a patient record 172 of the current patient) or reject the record recommendation 236 (e.g., to indicate that the record recommendation 236 is not accurate and should not be included in the patient record 172).

When the record recommendation 236 is confirmed, the record application 158 may use the AI model 115 (e.g., embodied as a learning action model) to add the information in the record recommendation 236 into the patient record 172 of the patient (e.g., by transforming/converting the information, storing the information accurately within the data structure of the patient record 172, etc.). The record application 158 may also transmit feedback data to the AI model 115 indicating the accuracy of the predicted record recommendation 236, such that the AI model 115 may adjust the model, weightings, and algorithm to generate more accurate predictions over time.

Similarly, when the confidence score 238 is greater than a predefined threshold, the medical application 160 may transmit, using the radio transceiver 161, the medical recommendation 150 to the second device 106 of the senior healthcare worker. The application 139 at the second device 106 may present the medical recommendation 150 (e.g., in human-readable or viewable form) for display on the display 141. The senior healthcare worker may select a user interface button/icon on the user interface 142 to either confirm the medical recommendation 150 (e.g., to indicate that the medical recommendation 150 is accurate and the task/action indicated in the medical recommendation 150 is to be performed by the junior healthcare worker) or reject the medical recommendation 150 (e.g., to indicate that the medical recommendation 150 is not accurate and the task/action indicated in the medical recommendation 150 should not be performed by the junior healthcare worker).

When the medical recommendation 150 is confirmed, the second device 106 may transmit, using the radio transceiver 143, the medical recommendation 150 to the first device 103 of the junior healthcare worker. The application 125 at the first device 103 may present the medical recommendation 150 or instructions associated with the actions/tasks in the medical recommendation 150 to perform with respect to the patient (e.g., in human-readable or viewable form) for display on the display 127. For example, the actions/tasks may be presented in the form of text, images, and/or videos (e.g., a combination of all three forms).

In some cases, the medical application 160 may use the AI model 115 (e.g., embodied as a learning action model) to actually perform one or more of the actions/tasks confirmed by the senior healthcare worker (e.g., automatically place an order for a confirmed prescription, automatically contact a recommended specialist healthcare worker, etc.). The medical application 160 may also transmit feedback data to the AI model 115 indicating the accuracy of the predicted medical recommendation 150, such that the AI model 115 may adjust the training, weighting, and/or prediction algorithms to improve the system to generate more accurate predictions.

Referring specifically now to FIG. 2C, shown is a block diagram illustrating the training of the AI model 115 and the use of the AI model 115 to generate instructions for equipment operations and provide medical education data. FIG. 2C specifically illustrates a method 250 performed by the medical application 160 of the healthcare facility system 109 to generate instructions for equipment operations and provide medical education data to a junior healthcare worker for transmission to a first device 103 of a junior healthcare worker.

Method 250 may begin with operation 252 to train the AI model 115 using the equipment data 146 indicating the instructions of operations for various types of medical equipment (e.g., including the medical devices 112) and to train the AI model using the medical education data 144 including general medical information used for healthcare worker education. For example, the medical application 160 may periodically or continuously transmit updates to the equipment data 146 and/or the education data 144 to the AI model 115. The AI model 115 may use the updates to continuously update the training, weights, and/or prediction algorithms of the AI model 115.

After and as the AI model 115 is continuously trained, the medical application 160 may use the AI model 115 to generate various types of instructions and educational training presentations to transmit to the first device 103 of the junior healthcare worker to assist in the patient care while providing patient-specific medical training to the junior healthcare worker. As mentioned above, the medical application 160 may use the AI model 115 to generate one or more medical recommendations 150, which are sent to the second device 106 of the senior healthcare worker for confirmation or rejection. For example, the senior healthcare worker may select the confirmed medical recommendations 150 via the user interface 142 of the second device 106. The medical application 160 may provide the confirmed medical recommendations 150 as input 257 into the AI model 115. The AI model 115 may run various methods/algorithms, developed based on the aforementioned training, to generate step-by-step instructions 262 and patient-specific medical education data 174, as output 258. For example, the medical application 160 may use the AI model 115 to obtain (e.g., generate or receive) step-by-step instructions 262 and patient-specific medical education data 174.

The step-by-step instructions 262 may be specific to a task/action indicated in the confirmed medical recommendation 150 that is to be performed by the junior healthcare worker with respect to the patient. The step-by-step instructions 262 may be instructions for operating one or more pieces of medical equipment to perform the task/action indicated in the confirmed medical recommendation 150. The step-by-step instructions 262 may be different for different types of patients, and as such, in some cases, the step-by-step instructions 262 predicted by the AI model 115 are tailored to the needs and the conditions of the current patient being treated by the junior healthcare worker. In this case, the medical data 204 may have also been provided as input 257 into the AI model 115 (if not previously done for the other predictions described above with reference to FIGS. 2A-B).

The patient-specific medical education data 174 may also be specific to a task/action indicated in the confirmed medical recommendation 150 that is to be performed by the junior healthcare worker with respect to the patient. For example, the patient-specific medical education data 174 may include general medical information describing the conditions (e.g., when, why, how, etc.) of the patient that may be the reason behind performing the task/action indicated in the confirmed medical recommendation 150. For example, the patient-specific medical education data 174 may also include information regarding issues or problems that may occur or be experienced by the patient as a result of performing the task/action, and methods of potentially avoiding such issues or problems.

The medical application 160 may transmit, using the radio transceiver 161, the step-by-step instructions 262 and/or the patient-specific medical education data 174 to one or more first devices 103 of the junior healthcare worker. The application 125 at the first device 103 may present information in the step-by-step instructions 262 and/or the patient-specific medical education data 174 (e.g., in human-readable or viewable form) for display on the display 127. For example, the actions/tasks may be presented in the form of text, images, and/or videos (e.g., a combination of all three forms). The medical application 160 may also transmit feedback data to the AI model 115 indicating the accuracy of the predicted step-by-step instructions 262 and/or the patient-specific medical education data 174 (e.g., based on feedback received from the junior healthcare worker indicating whether the step-by-step instructions 262 and/or the patient-specific medical education data 174 was helpful or not). Once the feedback data is received, the AI model 115 may use the received feedback data to adjust the training, weighting, and/or prediction algorithms to improve the system to generate more accurate predictions.

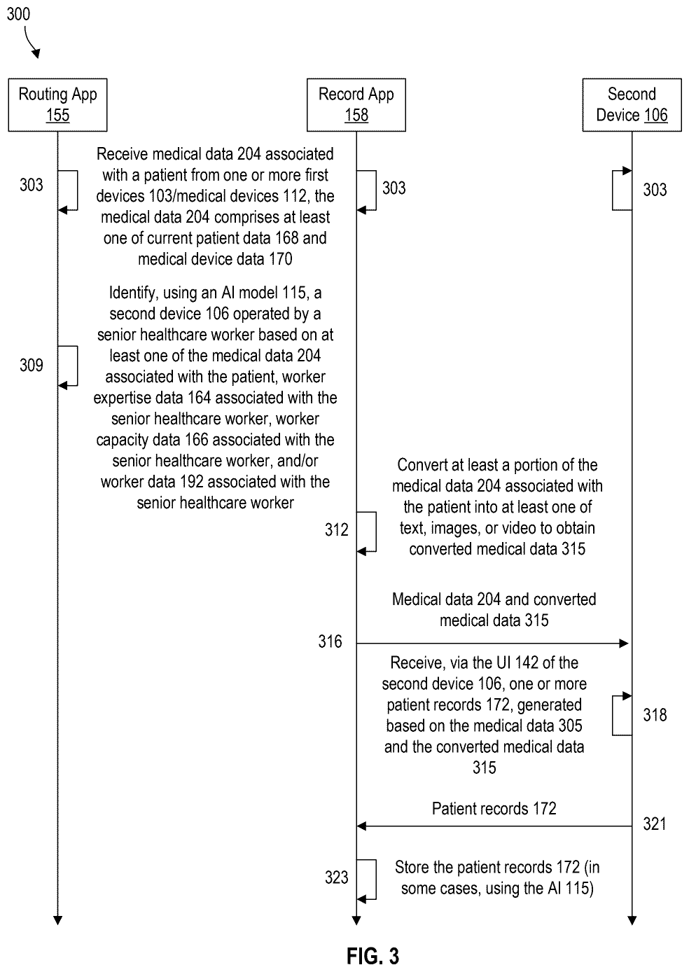

Referring now to FIG. 3, shown is a message sequence diagram illustrating a method 300 for using AI to enhance communications between healthcare workers for patient care and documentation according to various embodiments of the disclosure. Method 300 may be performed by the routing application 155 and the record application 158 of the healthcare facility system 109, and the second device 106 operated by the senior healthcare worker.

Method 300 may begin with operation 303, in which the routing application 155 and the record application 158 (and the medical application 160) each receive medical data 204 associated with a patient from one or more first devices 103 and/or one or medical devices 112 (e.g., via radio connections provided by the radio transceivers 134, 196). The medical data 204 comprises at least one of the current patient data 168 and the medical device data 170.

At operation 309, the routing application 155 may identify, using an AI model 115, a second device 106 operated by a senior healthcare worker based on at least one of the medical data 204 associated with the patient, worker expertise data 164 associated with the senior healthcare worker, worker capacity data 166 associated with the senior healthcare worker, or worker data 192 associated with the senior healthcare worker. This operation 309 may be performed similar to the operations described above with reference to FIG. 2A.

At operation 312, the record application 158 may convert at least a portion of the medical data 204 associated with the patient into at least one of text, images, or video to obtain converted medical data 315. For example, the medical data 204 may include a recording of a conversation between the junior healthcare worker and the patient, and the record application 158 may use, for example, a speech-to-text algorithm to convert the conversation into text.

At operation 316, the record application 158 may transmit the medical data 204 and the converted medical data 315 to the second device 106 associated with the senior healthcare worker. At operation 318, the second device 106 may receive, via the user interface 142 of the second device 106, one or more patient records 172. For example, the senior healthcare worker may manually type the information to be included in the patient record 172 for a patient into the second device 106. The senior healthcare worker may also dictate the information into a microphone of the second device 106, and the application 139 may use the AI model 115 to translate a recording of the dictation into text. In some cases, the record application 158 may use the AI model 115 to generate record recommendations 236, as described above with reference to FIG. 2B, which may be displayed at the second device 106 and confirmed/rejected by the senior healthcare worker. The application 139 at the second device 106 may add the confirmed record recommendations 236 to the patient record 179 for the patient.

At operation 321, the second device 106 may transmit the patient records 172 to the record application 158. At operation 323, the record application 158 may store the patient records 172 at the data store 162. In some cases, the second device 106 may also input the patient records 172 into the AI model 115, such that the AI model 115 automatically stores the patient records 172 at the data store 162 in an optimal and seamless manner.

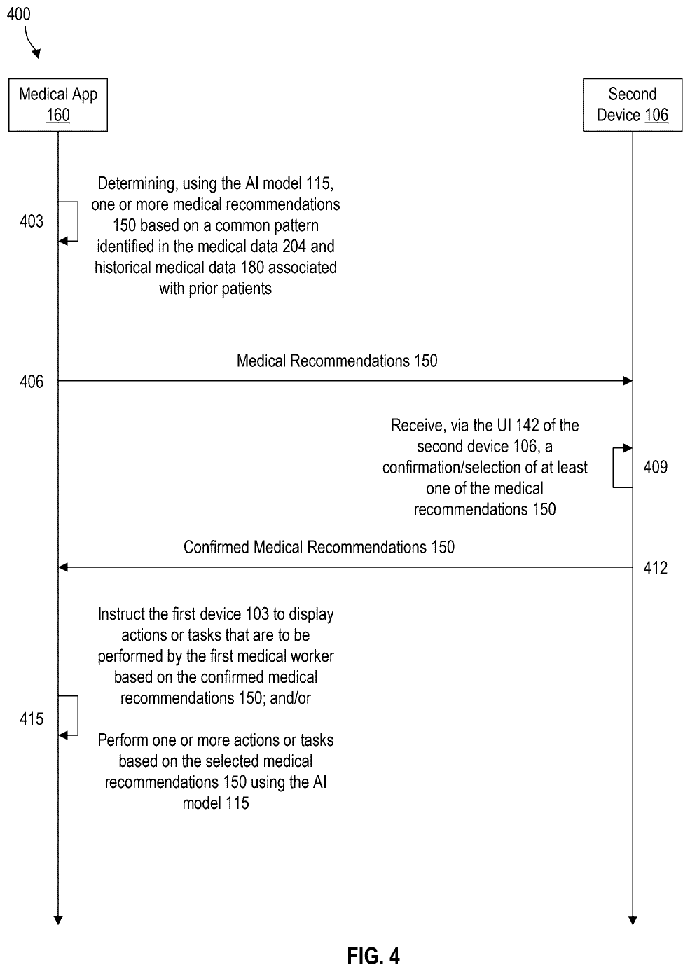

Referring now to FIG. 4, shown is a message sequence diagram illustrating a method 400 for using AI to enhance communications between healthcare workers for patient care and documentation according to various embodiments of the disclosure. Method 400 may be performed by the medical application 160 of the healthcare facility system 109 and the second device 106 operated by the senior healthcare worker.

Method 400 may begin with operation 403, in which the medical application 160 determines, using the AI model 115, one or more medical recommendations 150 based on a common pattern (e.g., correlation or trend) identified in the medical data 204 and the historical medical data 180 associated with multiple prior patients. This operation 403 may be performed similar to the operations described above with reference to FIG. 2B. At operation 406, the medical application 160 may transmit, using the radio transceiver 161, the medical recommendations 150 to the second device 106 (e.g., via a cellular radio connection between the healthcare facility system 109 and the second device 106).

At operation 409, the second device 106 may receive, via the user interface 142 of the second device 106, a confirmation (e.g., selection) of at least one of the medical recommendations 150 that are accurate and agreed to by the senior healthcare worker as indicating actions/tasks that ought to be performed by the junior healthcare worker with respect to the patient. At operation 412, the second device 106 transmits, to the medical application 160 of the healthcare facility system 109 using the radio transceiver 143, the confirmed medical recommendations 150 (which may include one or more of the medical recommendations 150 initially sent to the second device 106). For example, the confirmed medical recommendations 150 may be transmitted to the medical application 160 via a cellular radio connection between the healthcare facility system 109 and the second device 106.

At operation 415, the medical application 160 may transmit instructions to the first device 103 to display actions or tasks that are to be performed by the junior healthcare worker based on the confirmed medical recommendations 150. In addition or alternatively, at operation 415, the medical application 160 may automatically perform one or more of the actions or tasks based on the confirmed medical recommendations using the AI model 115 (e.g., embodied as a learning action model).

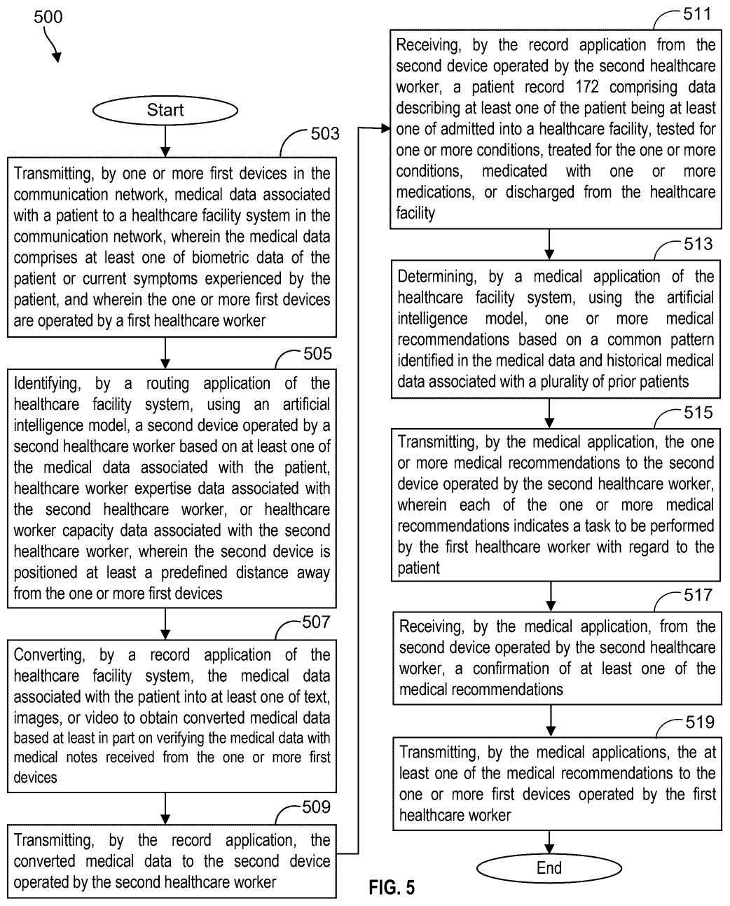

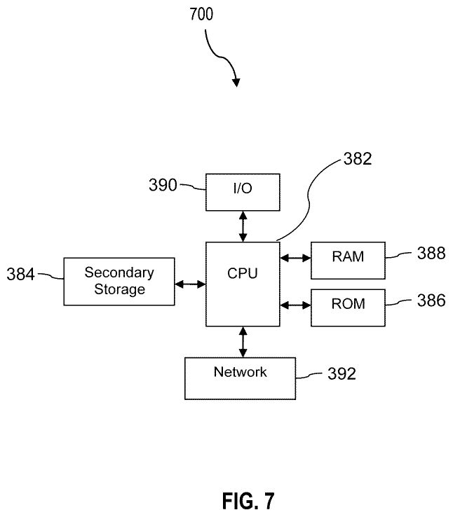

Referring now to FIG. 5, shown is a method 500 of using AI to enhance communications between healthcare workers for patient care and documentation according to various embodiments of the disclosure. Method 500 may be performed by routing application 155, record application 158, and/or medical application 160 of the healthcare facility system 109, the application 125 across one or more first devices 103 operated by a junior healthcare worker, and/or the application 139 of a second device 106 operated by a senior healthcare worker. Hereinafter, the junior healthcare worker may also be referred to as a “first healthcare worker,” while the senior healthcare worker may also be referred to as a “second healthcare worker.” In embodiments, the method 500 may be implemented using a computer system with components as shown in FIG. 7. As illustrated, method 500 of FIG. 5 includes a number of enumerated operations, but embodiments of the operations in FIG. 5 may include additional operations before, after, and in between the enumerated operations. In some embodiments, one or more of the enumerated operations may be omitted or performed in a different order.

At step 503, method 500 may comprise transmitting, by one or more first devices 103 in the communication network 100, medical data 204 associated with a patient to a healthcare facility system 109 in the communication network 100. The medical data 204 comprises at least one of biometric data of the patient or current symptoms experienced by the patient (e.g., as indicated in the current patient data 168). The one or more first devices 103 are operated by a first healthcare worker (e.g., a junior healthcare worker).

At step 505, method 500 may comprise identifying, by a routing application 155 of the healthcare facility system 109, using an AI model 115, a second device 106 operated by a second healthcare worker based on at least one of the medical data 204 associated with the patient, healthcare worker expertise data 164 associated with the second healthcare worker, or healthcare worker capacity data 166 associated with the second healthcare worker. The second device 106 is positioned at least a predefined distance 153 away from the one or more first devices 103.

At step 507, method 500 may comprise converting, by a record application 158 of the healthcare facility system 109, the medical data 204 associated with the patient into at least one of text, images, or video to obtain converted medical data 315 based at least in part on verifying the medical data with medical notes received from the one or more first devices operated by the first healthcare worker. At step 509, method 500 may comprise transmitting, by the record application 158, the converted medical data 315 to the second device 106 operated by the second healthcare worker.

At step 511, method 500 may comprise receiving, by the record application 158 from the second device 106 operated by the second healthcare worker, a patient record 172 comprising data describing the patient being at least one of admitted into a healthcare facility, tested for one or more conditions, treated for the one or more conditions, medicated with one or more medications, or discharged from the healthcare facility. At step 513, method 500 may comprise determining, by a medical application 160 of the healthcare facility system 109, using the AI model 115, one or more medical recommendations 150 based on a common pattern identified in the medical data 204 and historical medical data 180 associated with a plurality of prior patients. At step 515, method 500 may comprise transmitting, by the medical application 160, the one or more medical recommendations 150 to the second device 106 operated by the second healthcare worker. Each of the one or more medical recommendations 150 indicates a task to be performed by the first healthcare worker with regard to the patient.

At step 517, method 500 may comprise receiving, by the medical application 160, from the second device 106 operated by the second healthcare worker, a confirmation of at least one of the medical recommendations. At step 519, method 500 may comprise transmitting, by the medical applications 160, the at least one of the medical recommendations 150 to the one or more first devices 103 operated by the first healthcare worker.

Method 500 may include other steps and/or features that are not otherwise shown in FIG. 5. In an embodiment, wherein the one or more first devices 103 comprise at least one of a portable handheld device or a wearable device, and the one or more first devices 103 comprise a radio transceiver 143. In an embodiment, the biometric data comprises at least one of a heart rate, a blood pressure, a respiratory rate, or a temperature of the patient, and the biometric data is obtained from one or more medical devices 112 configured to sense the biometric data of the patient.

In an embodiment, method 500 may further comprise obtaining, by the one or more first devices 103, the medical data 204 associated with the patient by either receiving the medical data 204 as input from the first healthcare worker via a user interface 129 of the one or more first devices 103, or receiving the medical data 204 via a radio transceiver 143 of the one or more first devices 103 from one or more medical devices 112 configured to collect data from the patient. In an embodiment, converting, by the record application 158, the medical data 204 associated with the patient to the at least one of text, images, or video to obtain the converted medical data 315 comprises converting, by the record application 158, an audio recording of a conversation between the first healthcare worker and the patient into text using a voice biometrics application.

In an embodiment, the historical medical data 180 comprises at least one of symptoms data 186, diagnosis data 188, or treatment data 190 associated with the prior patients, in which the diagnosis data 188 indicates at least one of a test performed on the prior patients or a confirmed diagnosis of the prior patients, and the treatment data 190 indicates at least one of medicines administered to the prior patients or procedures performed on the prior patients. In an embodiment, the one or more medical recommendations 150 further comprise step-by-step instructions 262 for using medical equipment to perform the task, wherein the task comprises at least one of a test or a procedure to be performed on the patient.

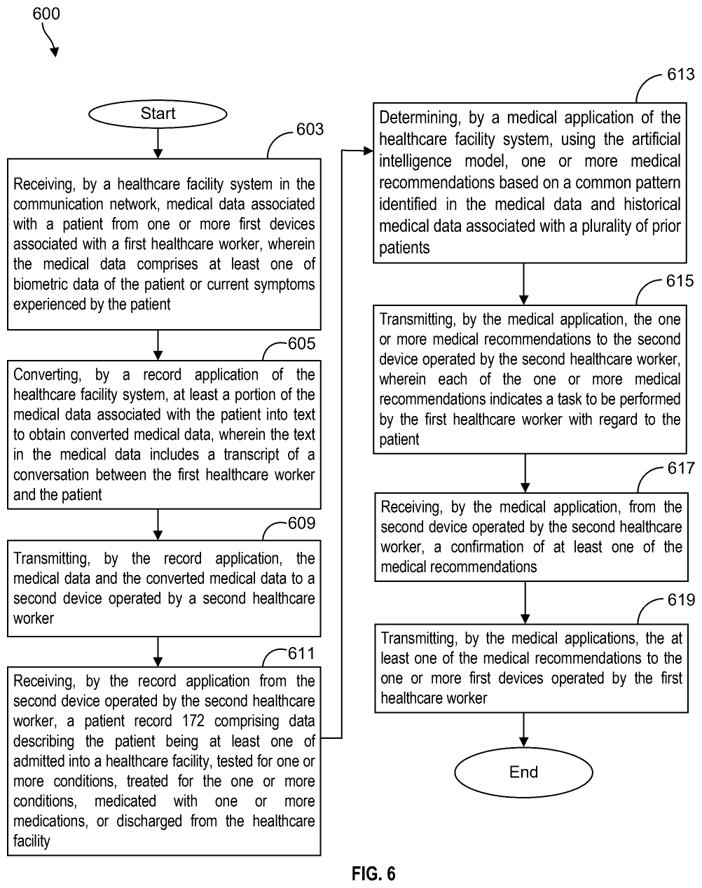

Referring now to FIG. 6, shown is a method 600 of using AI to enhance communications between healthcare workers for patient care and documentation according to various embodiments of the disclosure. Method 600 may be performed by routing application 155, record application 158, and/or medical application 160 of the healthcare facility system 109, the application 125 across one or more first devices 103 operated by a junior healthcare worker, and/or the application 139 of a second device 106 operated by a senior healthcare worker. Hereinafter, the junior healthcare worker may also be referred to as a “first healthcare worker,” while the senior healthcare worker may also be referred to as a “second healthcare worker.” In embodiments, the method 600 may be implemented using a computer system with components as shown in FIG. 7. As illustrated, method 600 of FIG. 6 includes a number of enumerated operations, but embodiments of the operations in FIG. 6 may include additional operations before, after, and in between the enumerated operations. In some embodiments, one or more of the enumerated operations may be omitted or performed in a different order.

At step 603, method 600 may comprise receiving, by a healthcare facility system 109 in the communication network 100, medical data 204 associated with a patient. The medical data 204 comprises at least one of biometric data of the patient or current symptoms experienced by the patient (e.g., as indicated in the current patient data 168). The one or more first devices 103 are operated by a first healthcare worker (e.g., a junior healthcare worker).

At step 605, method 600 may comprise converting, by a record application 158 of the healthcare facility system 109, at least a portion the medical data 204 associated with the patient into text to obtain converted medical data 315. At step 609, method 600 may comprise transmitting, by the record application 158, the medical data 204 and the converted medical data 315 to the second device 106 operated by the second healthcare worker.

At step 611, method 600 may comprise receiving, by the record application 158 from the second device 106 operated by the second healthcare worker, a patient record 172 comprising data describing the patient being at least one of admitted into a healthcare facility, tested for one or more conditions, treated for the one or more conditions, medicated with one or more medications, or discharged from the healthcare facility. At step 613, method 600 may comprise determining, by a medical application 160 of the healthcare facility system 109, using the AI model 115, one or more medical recommendations 150 based on a common pattern identified in the medical data 204 and historical medical data 180 associated with a plurality of prior patients. At step 615, method 600 may comprise transmitting, by the medical application 160, the one or more medical recommendations 150 to the second device 106 operated by the second healthcare worker. Each of the one or more medical recommendations 150 indicates a task to be performed by the first healthcare worker with regard to the patient.

At step 617, method 600 may comprise receiving, by the medical application 160, from the second device 106 operated by the second healthcare worker, a confirmation of at least one of the medical recommendations. At step 619, method 600 may comprise transmitting, by the medical applications 160, the at least one of the medical recommendations 150 to the one or more first devices 103 operated by the first healthcare worker.

Method 600 may include other steps and/or features that are not otherwise shown in FIG. 6. In an embodiment, method 600 may further comprise identifying, by a routing application 155 of the healthcare facility system, using the AI model 115, the second device 106 operated by the second healthcare worker based on at least one of the medical data 204 associated with the patient, healthcare worker expertise data 164 associated with the second healthcare worker, or healthcare worker capacity data 166 associated with the second healthcare worker, in which the second device 106 is positioned at least a predefined distance 153 away from the one or more first devices 103.

In an embodiment, the one or more first devices 103 comprise at least one of a portable handheld device or a wearable device, and the one or more first devices comprise a radio transceiver 143. In an embodiment, the biometric data comprises at least one of a heart rate, a blood pressure, a respiratory rate, or a temperature of the patient, and the biometric data is obtained from one or more medical devices 112 configured to obtain the biometric data of the patient.

In an embodiment, the historical medical data 180 comprises at least one of symptoms data 186, diagnosis data 188, or treatment data 190 associated with the prior patients, in which the diagnosis data 188 indicates at least one of a test performed on the prior patients or a confirmed diagnosis of the prior patients, and the treatment data 190 indicates at least one of medicines administered to the prior patients or procedures performed on the prior patients. In an embodiment, the one or more medical recommendations 150 further comprise step-by-step instructions 262 for using medical equipment to perform the task, wherein the task comprises at least one of a test or a procedure to be performed on the patient. In an embodiment, the one or more medical recommendations 150 further comprise patient-specific medical education data 174 describing medical conditions associated with the at least one of the medical recommendations. In an embodiment, in response to receiving the confirmation of the at least one of the medical recommendations 150, method 600 may further comprise automatically, by the medical application 160, performing the task using the AI model 115.

FIG. 7 illustrates a computer system 700 suitable for implementing one or more embodiments disclosed herein. In an embodiment, first devices 103, second device 106, AI model 115, medical devices 112, and/or healthcare facility system 109 may each be implemented as the computer system 700. The computer system 700 includes a processor 382 (which may be referred to as a central processor unit or CPU) that is in communication with memory devices including secondary storage 384, read only memory (ROM) 386, random access memory (RAM) 388, input/output (I/O) devices 390, and network connectivity devices 392. The processor 382 may be implemented as one or more CPU chips.

It is understood that by programming and/or loading executable instructions onto the computer system 700, at least one of the CPU 382, the RAM 388, and the ROM 386 are changed, transforming the computer system 700 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 700 is turned on or booted, the CPU 382 may execute a computer program or application. For example, the CPU 382 may execute software or firmware stored in the ROM 386 or stored in the RAM 388. In some cases, on boot and/or when the application is initiated, the CPU 382 may copy the application or portions of the application from the secondary storage 384 to the RAM 388 or to memory space within the CPU 382 itself, and the CPU 382 may then execute instructions that the application is comprised of. In some cases, the CPU 382 may copy the application or portions of the application from memory accessed via the network connectivity devices 392 or via the I/O devices 390 to the RAM 388 or to memory space within the CPU 382, and the CPU 382 may then execute instructions that the application is comprised of. During execution, an application may load instructions into the CPU 382, for example load some of the instructions of the application into a cache of the CPU 382. In some contexts, an application that is executed may be said to configure the CPU 382 to do something, e.g., to configure the CPU 382 to perform the function or functions promoted by the subject application. When the CPU 382 is configured in this way by the application, the CPU 382 becomes a specific purpose computer or a specific purpose machine.

The secondary storage 384 is typically comprised of one or more disk drives or tape drives and is used for non-volatile storage of data and as an over-flow data storage device if RAM 388 is not large enough to hold all working data. Secondary storage 384 may be used to store programs which are loaded into RAM 388 when such programs are selected for execution. The ROM 386 is used to store instructions and perhaps data which are read during program execution. ROM 386 is a non-volatile memory device which typically has a small memory capacity relative to the larger memory capacity of secondary storage 384. The RAM 388 is used to store volatile data and perhaps to store instructions. Access to both ROM 386 and RAM 388 is typically faster than to secondary storage 384. The secondary storage 384, the RAM 388, and/or the ROM 386 may be referred to in some contexts as computer readable storage media and/or non-transitory computer readable media.

I/O devices 390 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 392 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards, and/or other well-known network devices. The network connectivity devices 392 may provide wired communication links and/or wireless communication links (e.g., a first network connectivity device 392 may provide a wired communication link and a second network connectivity device 392 may provide a wireless communication link). Wired communication links may be provided in accordance with Ethernet (IEEE 802.3), Internet protocol (IP), time division multiplex (TDM), data over cable service interface specification (DOCSIS), wavelength division multiplexing (WDM), and/or the like. In an embodiment, the radio transceiver cards may provide wireless communication links using protocols such as code division multiple access (CDMA), global system for mobile communications (GSM), long-term evolution (LTE), WiFi (IEEE 802.11), Bluetooth, Zigbee, narrowband Internet of things (NB IoT), near field communications (NFC), and radio frequency identity (RFID). The radio transceiver cards may promote radio communications using 5G, 5G New Radio, or 5G LTE radio communication protocols. These network connectivity devices 392 may enable the processor 382 to communicate with the Internet or one or more intranets. With such a network connection, it is contemplated that the processor 382 might receive information from the network, or might output information to the network in the course of performing the above-described method steps. Such information, which is often represented as a sequence of instructions to be executed using processor 382, may be received from and outputted to the network, for example, in the form of a computer data signal embodied in a carrier wave.

Such information, which may include data or instructions to be executed using processor 382 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well-known to one skilled in the art. The baseband signal and/or signal embedded in the carrier wave may be referred to in some contexts as a transitory signal.

The processor 382 executes instructions, codes, computer programs, scripts which it accesses from hard disk, floppy disk, optical disk (these various disk based systems may all be considered secondary storage 384), flash drive, ROM 386, RAM 388, or the network connectivity devices 392. While only one processor 382 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 384, for example, hard drives, floppy disks, optical disks, and/or other device, the ROM 386, and/or the RAM 388 may be referred to in some contexts as non-transitory instructions and/or non-transitory information.

In an embodiment, the computer system 700 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 700 to provide the functionality of a number of servers that is not directly bound to the number of computers in the computer system 700. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 700, at least portions of the contents of the computer program product to the secondary storage 384, to the ROM 386, to the RAM 388, and/or to other non-volatile memory and volatile memory of the computer system 700. The processor 382 may process the executable instructions and/or data structures in part by directly accessing the computer program product, for example by reading from a CD-ROM disk inserted into a disk drive peripheral of the computer system 700. Alternatively, the processor 382 may process the executable instructions and/or data structures by remotely accessing the computer program product, for example by downloading the executable instructions and/or data structures from a remote server through the network connectivity devices 392. The computer program product may comprise instructions that promote the loading and/or copying of data, data structures, files, and/or executable instructions to the secondary storage 384, to the ROM 386, to the RAM 388, and/or to other non-volatile memory and volatile memory of the computer system 700.

In some contexts, the secondary storage 384, the ROM 386, and the RAM 388 may be referred to as a non-transitory computer readable medium or a computer readable storage media. A dynamic RAM embodiment of the RAM 388, likewise, may be referred to as a non-transitory computer readable medium in that while the dynamic RAM receives electrical power and is operated in accordance with its design, for example during a period of time during which the computer system 700 is turned on and operational, the dynamic RAM stores information that is written to it. Similarly, the processor 382 may comprise an internal RAM, an internal ROM, a cache memory, and/or other internal non-transitory storage blocks, sections, or components that may be referred to in some contexts as non-transitory computer readable media or computer readable storage media.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method implemented in a communication network including to provide artificial intelligence enhanced communications between healthcare workers for patient care and documentation, wherein the method comprises:
transmitting, by one or more first devices in the communication network, medical data associated with a patient to a healthcare facility system in the communication network, wherein the medical data comprises at least one of biometric data of the patient or current symptoms experienced by the patient, and wherein the one or more first devices are operated by a first healthcare worker;
identifying, by a routing application of the healthcare facility system, using an artificial intelligence model, a second device operated by a second healthcare worker based on at least one of the medical data associated with the patient, healthcare worker expertise data associated with the second healthcare worker, or healthcare worker capacity data associated with the second healthcare worker, wherein the second device is positioned at least a predefined distance away from the one or more first devices;
converting, by a record application of the healthcare facility system, the medical data associated with the patient into at least one of text, images, or video to obtain converted medical data based at least in part on verifying the medical data with medical notes received from the one or more first devices;
transmitting, by the record application, the converted medical data to the second device operated by the second healthcare worker;
receiving, by the record application from the second device operated by the second healthcare worker, records comprising data describing at least one of the patient being at least one of admitted into a healthcare facility, tested for one or more conditions, treated for the one or more conditions, medicated with one or more medications, or discharged from the healthcare facility;
determining, by a medical application of the healthcare facility system, using the artificial intelligence model, one or more medical recommendations based on a common pattern identified in the medical data and historical medical data associated with a plurality of prior patients;
transmitting, by the medical application, the one or more medical recommendations to the second device operated by the second healthcare worker, wherein each of the one or more medical recommendations indicates a task to be performed by the first healthcare worker with regard to the patient;
receiving, by the medical application, from the second device operated by the second healthcare worker, a confirmation of at least one of the medical recommendations; and
transmitting, by the medical applications, the at least one of the medical recommendations to the one or more first devices operated by the first healthcare worker.

2. The method of claim 1, wherein the one or more first devices comprise at least one of a portable handheld device or a wearable device, and wherein the one or more first devices comprise a radio transceiver.

3. The method of claim 1, wherein the biometric data comprises at least one of a heart rate, a blood pressure, a respiratory rate, or a temperature of the patient, and wherein the biometric data is obtained from one or more medical devices configured to sense the biometric data of the patient.

4. The method of claim 1, further comprising obtaining, by the one or more first devices, the medical data associated with the patient by either:
receiving the medical data as input from the first healthcare worker via a user interface of the one or more first devices; or
receiving the medical data via a radio transceiver of the one or more first devices from one or more medical devices configured to collect data from the patient.

5. The method of claim 1, wherein converting, by the record application, the medical data associated with the patient to at least one of text, images, or video to obtain the converted medical data comprises converting, by the record application, an audio recording of a conversation between the first healthcare worker and the patient into text using a voice biometrics application.

6. The method of claim 1, wherein the historical medical data comprises at least one of symptoms data, diagnosis data, or treatment data associated with the prior patients, wherein the diagnosis data indicates at least one of a test performed on the prior patients or a confirmed diagnosis of the prior patients, and wherein the treatment data indicates at least one of medicines administered to the prior patients or procedures performed on the prior patients.

7. The method of claim 1, wherein the one or more medical recommendations further comprise step-by-step instructions for using medical equipment to perform the task, wherein the task comprises at least one of a test or a procedure to be performed on the patient.

8. A method implemented in a communication network including to provide artificial intelligence enhanced communications between healthcare workers for patient care and documentation, wherein the method comprises:
receiving, by a healthcare facility system in the communication network, medical data associated with a patient from one or more first devices associated with a first healthcare worker, wherein the medical data comprises at least one of biometric data of the patient or current symptoms experienced by the patient;
converting, by a record application of the healthcare facility system, at least a portion of the medical data associated with the patient into text to obtain converted medical data, wherein the text in the medical data includes a transcript of a conversation between the first healthcare worker and the patient;
transmitting, by the record application, the medical data and the converted medical data to a second device operated by a second healthcare worker;
receiving, by the record application from the second device operated by the second healthcare worker, a patient record comprising data describing the patient being at least one of admitted into a healthcare facility, tested for one or more conditions, treated for the one or more conditions, medicated with one or more medications, or discharged from the healthcare facility;
determining, by a medical application of the healthcare facility system, using an artificial intelligence model, one or more medical recommendations based on a common pattern identified in the medical data and historical medical data associated with a plurality of prior patients;
transmitting, by the medical application to the second device operated by the second healthcare worker, the one or more medical recommendations, wherein each of the one or more medical recommendations represents a task to be performed by the first healthcare worker with regard to the patient;
receiving, by the medical application, from the second device operated by the second healthcare worker, a confirmation of at least one of the medical recommendations; and
transmitting, by the medical application, the at least one of the medical recommendations to the one or more first devices operated by the first healthcare worker.

9. The method of claim 8, further comprising identifying, by a routing application of the healthcare facility system, using the artificial intelligence model, the second device operated by the second healthcare worker based on at least one of the medical data associated with the patient, healthcare worker expertise data associated with the second healthcare worker, or healthcare worker capacity data associated with the second healthcare worker, wherein the second device is positioned at least a predefined distance away from the one or more first devices.

10. The method of claim 8, wherein the one or more first devices comprise at least one of a portable handheld device or a wearable device, and wherein the one or more first devices comprise a radio transceiver.

11. The method of claim 8, wherein the biometric data comprises at least one of a heart rate, a blood pressure, a respiratory rate, or a temperature of the patient, and wherein the biometric data is obtained from one or more medical devices configured to obtain the biometric data of the patient.

12. The method of claim 8, wherein the historical medical data comprises at least one of symptoms data, diagnosis data, or treatment data associated with the prior patients, wherein the diagnosis data indicates at least one of a test performed on the prior patients or a confirmed diagnosis of the prior patients, and wherein the treatment data indicates at least one of medicines administered to the prior patients or procedures performed on the prior patients.

13. The method of claim 8, wherein the one or more medical recommendations further comprise step-by-step instructions for using medical equipment to perform the task, wherein the task comprises at least one of a test or a procedure to be performed on the patient.

14. The method of claim 8, wherein the one or more medical recommendations further comprise educational training data describing medical conditions associated with the at least one of the medical recommendations.

15. The method of claim 8, wherein, in response to receiving the confirmation of the at least one of the medical recommendations, the method further comprises automatically, by the medical application, performing the task using the artificial intelligence model.

16. A healthcare facility system, comprising:
a non-transitory memory;
a processor coupled to the non-transitory memory;
a record application stored at the non-transitory memory, which when executed by the processor, causes the processor to be configured to:
receive medical data associated with a patient from one or more first devices associated with a first healthcare worker, wherein the medical data comprises at least one of biometric data of the patient or current symptoms experienced by the patient;
transmit the medical data to a second device operated by a second healthcare worker; and
receive, from the second device operated by the second healthcare worker, a patient record comprising data describing the patient being at least one of admitted into a healthcare facility, tested for one or more conditions, treated for the one or more conditions, medicated with one or more medications, or discharged from the healthcare facility;
a medical application stored at the non-transitory memory, which when executed by the processor, causes the processor to be configured to:
determine, using an artificial intelligence model, one or more medical recommendations based on a common pattern identified in the medical data and historical medical data associated with a plurality of prior patients;
transmit the one or more medical recommendations to the second device operated by the second healthcare worker, wherein each of the one or more medical recommendations represents a task to be performed by the first healthcare worker with respect to the patient; and
automatically perform at least one of the one or more medical recommendations in response to a confirmation received from the second device to perform the at least one of the one or more medical recommendations.

17. The healthcare facility system of claim 16, further comprising a routing application stored at the non-transitory memory, which when executed by the processor, causes the processor to be configured to identify, using the artificial intelligence model, the second device operated by the second healthcare worker based on at least one of the medical data associated with the patient, healthcare worker expertise data associated with the second healthcare worker, or healthcare worker capacity data associated with the second healthcare worker, wherein the second device is positioned at least a predefined distance away from the one or more first devices.

18. The healthcare facility system of claim 16, wherein the one or more first devices comprise at least one of a portable handheld device or a wearable device, and wherein the one or more first devices comprise a radio transceiver.

19. The healthcare facility system of claim 16, wherein the biometric data comprises at least one of a heart rate, a blood pressure, a respiratory rate, or a temperature of the patient.

20. The healthcare facility system of claim 16, wherein the historical medical data comprises at least one of symptoms data, diagnosis data, or treatment data associated with the prior patients, wherein the diagnosis data indicates at least one of a test performed on the prior patients or a confirmed diagnosis of the prior patients, and wherein the treatment data indicates at least one of medicines administered to the prior patients or procedures performed on the prior patients.
